(19) United States
(12) Patent Application Publication
(10) Pub. No.: US 2005/0281742 A1
(43) Pub. Date: Dec. 22, 2005

(54) Method of determining an individual's level of tolerance to application of a product to skin of the individual, and a kit for implementing such a method

(75) Inventor: Roland Bazin, Bievres (FR)

Correspondence Address: OLIFF & BERRIDGE, PLC, P.O. BOX 19928, ALEXANDRIA, VA, 22320, US

(73) Assignee: L'OREAL, Paris (FR)

(21) Appl. No.: 11/154,635

(22) Filed: Jun. 17, 2005

Related U.S. Application Data
(60) Provisional application No. 60/584,500, filed on Jul. 2, 2004.

(30) Foreign Application Priority Data
Jun. 18, 2004 (FR) 0451296

(51) Int. Cl. A61K 49/00

(52) U.S. Cl. 424/9.1

(57) ABSTRACT

A method of determining an individual's level of tolerance to application of a cosmetic product to skin of the individual may include: applying a substance onto a test zone of the skin, the substance containing at least one agent for stimulating the peripheral nervous system; exerting a mechanical action on the test zone of the skin to reduce a barrier function of the skin relative to the substance; and observing any reaction of the individual as a result of applying the substance. The substance may be the product itself and/or may be configured to produce a reaction representative of an undesirable reaction that might be observed after application of the product. The mechanical action may be exerted at least one of before, during and after application of the test substance.

### CROSS-REFERENCE TO RELATED APPLICATIONS

This non-provisional application claims the benefit of French Application No. 04 51296 filed on Jun. 18, 2004, and U.S. Provisional Application No. 60/584,500 filed on Jul. 2, 2004.

### BACKGROUND

The present invention relates to tests for determining an individual's level of tolerance to the application of a product on the skin, for example, a cosmetic product.

The term “cosmetic product” is used in the present description in the meaning defined in the Jun. 14, 1993 Directive 93/35/EEC modifying Directive 76/768/EEC.

### SUMMARY

There exists a need to enable consumers to have products available that exert a stronger action to obtain results more quickly and/or more markedly.

Nevertheless, with such products, there is a greater risk of application leading to an undesirable reaction with certain individuals, for example, those individuals having skin that is particularly sensitive.

Exemplary embodiments of the invention seek to enable an individual, prior to applying a product, to determine that individual's own level of tolerance to the product, for example, for the purpose of matching a “dosage” of the product to a degree of skin sensitivity, or to change to some other product. The term “dosage” is used herein to cover a quantity of product that is applied to the skin, a frequency of application, a concentration of agents of the product, and/or a nature of active agents of the product.

Exemplary embodiments of the invention provide a method of determining an individual's level of tolerance to application of a product on skin of the individual, the method comprising: applying a substance onto a test zone of the skin, the substance containing at least one agent for stimulating the peripheral nervous system, said substance at least one of comprising said product and configured to produce a reaction representative of an undesirable reaction that might be observed after application of said product; exerting a mechanical action on the test zone of the skin for reducing a barrier function of the skin relative to the substance, said action being exerted at least one of before, during and after application of the test substance; and observing any reaction of the individual as a result of applying the substance.

The term “agent for stimulating the peripheral nervous system” is used herein to designate an agent capable of inducing a sensory response associated with activating sensitive cutaneous nerves with terminations flush with the level of the stratum corneum.

For example, exerting a mechanical action may comprise exerting a peeling action by applying and then removing an adhesive member. Exerting a mechanical action may also comprise exerting an abrasive action.

In exemplary embodiments, the mechanical action may advantageously be exerted prior to applying the substance.

In exemplary embodiments, the substance may contain, for example, at least one of capsaicin, lactic acid, alcohol and sodium chloride.

In exemplary embodiments, the skin may preferably be cleaned before applying the substance and also before exerting the mechanical action on the skin.

In exemplary embodiments, it may be preferable to perform an action of rinsing the test zone at an end of the test.

In exemplary embodiments, a product dosage may advantageously be determined as a function of a reaction that is observed. The reaction may, for example, comprise itching, a burning sensation, and/or a stinging sensation.

In exemplary embodiments, the product may comprise an antiwrinkle agent, a peel, for example, containing alpha-hydroxy acids and/or abrasive fillers, a product for stimulating renewal of epidermis, a slimming product, an anti-acne product, a depilatory product, a sunscreen, a makeup and/or a care product, this list not being exhaustive.

In exemplary embodiments, the substance may preferably be applied on a sensitive zone, for example, on the face of the individual, such as on the nasolabial fold.

Exemplary embodiments of the invention provide a kit enabling an individual's level of tolerance to application of a product on skin of the individual to be determined, the kit comprising: a substance that contains at least one agent for stimulating the peripheral nervous system; and a member configured to exert a mechanical action on the skin suitable for lowering a barrier function thereof relative to the substance.

Exemplary embodiments of the invention provide a kit enabling an individual's level of tolerance to application of a product on skin of the individual to be determined, the kit comprising: a substance that contains at least one agent for stimulating the peripheral nervous system; a member configured to exert a mechanical action on the skin suitable for lowering a barrier function thereof relative to the substance; and an applicator for the substance, said member being distinct from the applicator.

In exemplary embodiments, the member configured to exert a mechanical action on the skin may be at least one of adhesive and abrasive.

In exemplary embodiments, the kit may further include information useful in prescribing a product as a function of a reaction observed after applying the substance.

In exemplary embodiments, the substance may comprise the product to be applied on the skin after the individual's level of tolerance has been evaluated. In exemplary embodiments, the substance may also be different from the product to be applied on the skin after evaluating the individual's level of tolerance. For example, the substance may contain at least one of capsaicin and lactic acid.

Where appropriate or desired, the substance may be contained in a single-application package.

In exemplary embodiments, the kit may include a lotion for rinsing the test zone.

In exemplary embodiments, the kit may further include the product to be applied to the skin, for example, in the event of a positive evaluation of the individual's tolerance to said product.

Exemplary embodiments of the invention provide a method of prescribing a cosmetic, comprising: evaluating a degree of tolerance of the skin, by implementing the above-defined method; and prescribing at least one of a cosmetic and a dosage as a function of a result of the evaluation.

In exemplary embodiments, the prescription may take place at a point of sale, for example, in a department store, a beauty parlor or a dispensary, or remotely, with a consumer receiving the prescription by e-mail or by physical mail. In the case of physical mail, the mail may be accompanied by the prescribed product, where appropriate or desired.

### BRIEF DESCRIPTION OF THE DRAWINGS

The invention will be better understood on reading the following detailed description of non-limiting implementations thereof, and on examining the accompanying drawings, in which:

FIG. 1 is a diagram of an exemplary kit;

FIG. 2 shows an exemplary member for exerting mechanical action on the skin;

FIG. 3 is a block diagram showing various steps in an exemplary method;

FIG. 4 is an exemplary table suitable for evaluating a dosage to be used as a function of observed reactions; and

FIG. 5 is a diagram of an exemplary single-application packaging.

### DETAILED DESCRIPTION OF EXEMPLARY EMBODIMENTS

An exemplary kit 1 shown in FIG. 1 seeks to enable an individual's level of tolerance to application of a product on skin of the individual to be evaluated. The product may optionally be contained in the kit 1.

In the exemplary embodiment, the product for application to the skin may be included in the kit 1 and may be packaged, for example, in a tube 2. However, it should be understood that the product may be packaged in some other way, for example, in a pot, a flask, a receptacle fitted with an applicator, or in the form of a solid cake, this list not being limiting. The type of packaging may be selected as a function of a nature of the product and a region of the skin on which the product is to be applied.

The kit 1 may comprise a member 3 configured to exert a mechanical action on the skin. The mechanical action may seek to reduce a barrier function of the skin.

The kit 1 may also include a test substance which, when applied to the skin, may be suitable for stimulating the peripheral nervous system.

For example, the substance may comprise the product itself. However, the substance may differ from the product itself. For example, the substance may contain at least one agent for stimulating the peripheral nervous system, for example, capsaicin, lactic acid, alcohol, and/or sodium chloride.

In the exemplary embodiment, the substance may be contained in a single-application flask 5. However, it should be understood that the substance may be contained in any other form of packaging, for example, a tube of the cotton swab type provided with a liquid plug as described in patent application FR 2 845 005, or as sold by the supplier SWABPLUS®. A tube 15 sold by SWABPLUS® is shown diagrammatically in FIG. 5. The tube 15 includes a break-off end 16 (shown after being broken off) and an end 17 for applying the substance contained in the tube.

In the exemplary embodiment, the kit 1 may further comprise means for cleaning the skin prior to applying the test substance. The cleaning means may comprise, for example, by a wipe impregnated with a wetting agent and contained in a hermetically-sealed sachet 6.

Finally, in the exemplary embodiment, the kit I may also include instructions 8 containing information for determining a dosage for applying the product contained in the receptacle 2 as a function of a reaction observed after applying the test substance.

In exemplary embodiments, the instructions 8 may be replaced by any other information medium, for example, a label present on the packaging (not shown) containing the various elements of the kit.

In the exemplary embodiment, the member 3 configured to exert mechanical action on the skin may comprise a disk cut from an adhesive film 9, the disk being provided with a pull tongue 10 making it easier to detach from a remainder of the film 9. For example, the disk may be of the type sold under the reference Cornéofix by the supplier Monaderm (Monaco).

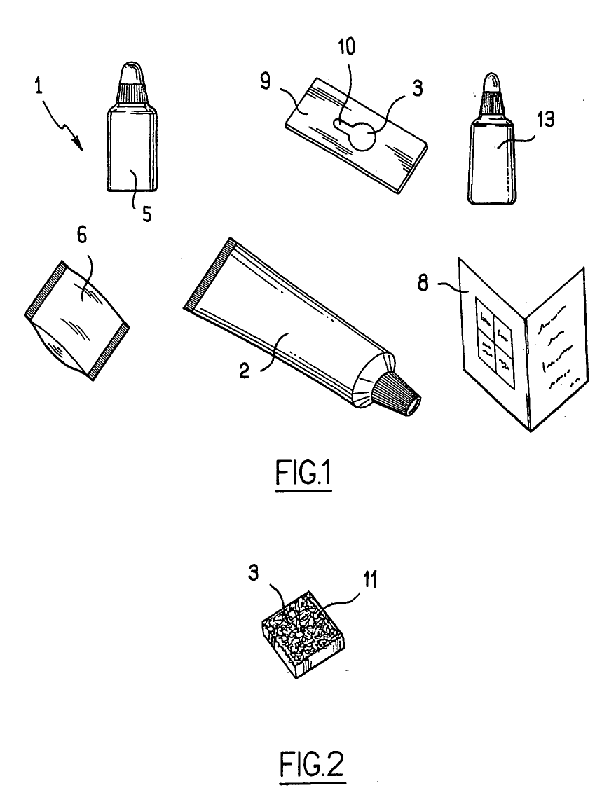

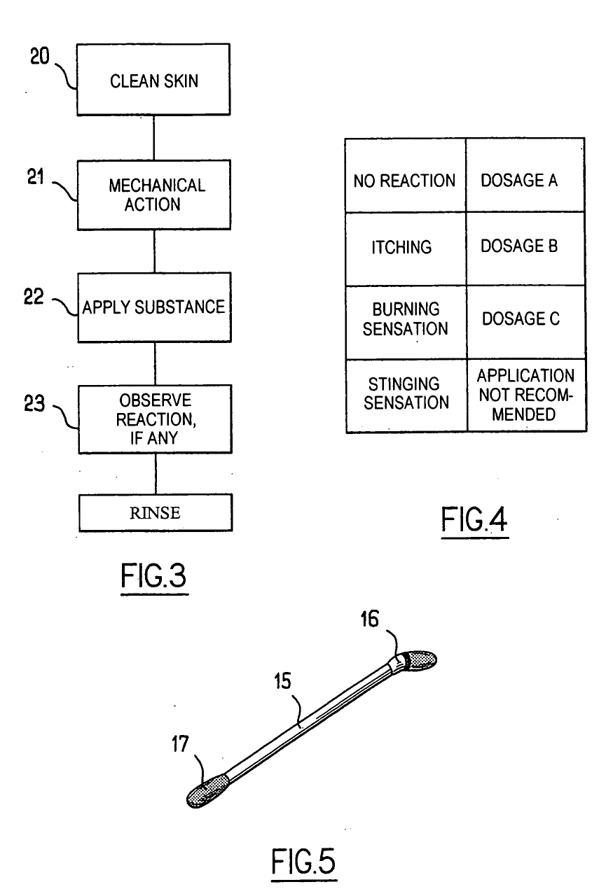

Other members may be used for exerting a mechanical action on the skin. For example, as shown in FIG. 2, a member 3 provided with an abrasive face 11 may be used.

In exemplary embodiments, the kit may be used as follows.

Firstly, a user may proceed in a first step 20 by cleaning a region of the individual's skin that is to be used for test purposes.

For example, the region may be part of the individual's face that is particularly sensitive, such as the nasolabial fold.

Cleaning may be performed, for example, using the wipe contained in the sachet 6.

Thereafter, once the test zone has been cleaned, mechanical action may be exerted in a step 21 using the adhesive member 3 of FIG. 1 or the abrasive member 3 of FIG. 2. When using an adhesive member, reduction in the barrier function of the skin may be a result of the adhesive member pulling away cells from a surface of the skin when the adhesive member is applied and then pulled off. With an abrasive member, reduction in the barrier function may be achieved by surface erosion action on the skin.

The mechanical action that is exerted may facilitate penetration of the substance during a step 22. The mechanical action may also sensitize the skin and amplify the stimulation exercised by the test substance.

After the substance has been applied in step 22, the user may proceed with a step 23 of observing any reaction.

There may be no reaction, or there may be a sensory reaction such as, for example, a reaction of itching, burning, and/or stinging.

For example, a burning sensation may be similar to that felt when burned by high temperature. A stinging sensation may, for example, be similar to that felt when alcohol is put on a cut.

The application of the test substance may also lead to a more or less intense red mark appearing in the test zone.

Based on the observed reaction, it may be possible to determine a suitable dosage for applying the product contained in the receptacle 2. For example, a table giving advice of the kind shown in FIG. 4 may be used to determine the suitable dosage. Such a table may put each observed reaction in correspondence with a recommended dosage.

For example, dosages may differ from one another in frequency of application, quantity of substance used, concentration of active agents, and/or a nature of active agents.

For example, when there is no reaction, daily application may be envisaged. When there is a light reaction, application once every two days may be indicated. When there is a burning sensation, application once a week may be indicated. When there is a stinging sensation, then application of the product may not be recommended.

Based on the observed reaction, different types of product may be prescribed.

At the end of the test, the test zone may preferably be rinsed, for example, using a suitable lotion contained in a flask 13 in the kit 1.

Naturally, the invention is not limited to the embodiments described above.

For example, the member configured to exert a mechanical action on the skin may be made in some other way.

Throughout the description, including in the claims, the term “comprising a” should be understood as being synonymous with “comprising at least one” unless specified to the contrary.

Although the present invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention.

1. A method of determining an individual's level of tolerance to application of a cosmetic product to skin of the individual, comprising:
applying a substance onto a test zone of the skin, the substance containing at least one agent for stimulating the peripheral nervous system, the substance at least one of comprising said product and configured to produce a reaction representative of an undesirable reaction that might be observed after application of said product;
exerting a mechanical action on the test zone of the skin for reducing a barrier function of the skin relative to the substance, said action being exerted at least one of before, during and after application of the test substance; and
observing any reaction of the individual as a result of applying the substance.

2. A method according to claim 1, wherein exerting a mechanical action comprises exerting a peeling action by applying and then removing an adhesive member.

3. A method according to claim 1, wherein exerting a mechanical action comprises exerting an abrasive action.

4. A method according to claim 1, wherein exerting a mechanical action is prior to applying the substance.

5. A method according to claim 1, wherein the substance contains at least one of capsaicin and lactic acid.

6. A method according to claim 1, further comprising cleaning the skin before applying the substance and after exerting the mechanical action.

7. A method according to claim 1, further comprising rinsing the test zone at an end of the test.

8. A method according to claim 1, further comprising determining a dosage for the product as a function of the observed reaction.

9. A method according to claim 1, wherein applying the substance comprises applying the substance to the face.

10. A method according to claim 9, wherein applying the substance comprises applying the substance to the nasolabial fold.

11. A kit enabling an individual's level of tolerance relative to application of a product on skin of the individual to be determined, comprising:
a substance that contains at least one agent for stimulating the peripheral nervous system; and
an adhesive member configured to exert a mechanical action on the skin that lowers a barrier function thereof relative to the substance.

12. A kit according to claim 11, wherein the substance comprises the product to be applied to the skin after evaluating the individual's level of tolerance to said product.

13. A kit according to claim 11, wherein the substance is different from the product to be applied to the skin after evaluating the individual's level of tolerance to said product.

14. A kit according to claim 13, wherein the substance contains at least one of capsaicin, lactic acid, alcohol and sodium chloride.

15. A kit according to claim 11, further comprising single-application packaging in which the substance is contained.

16. A kit according to claim 11, further comprising a lotion for rinsing the test zone.

17. A kit according to claim 11, further comprising the substance that is to be applied to the skin.

18. A kit according to claim 12, wherein the product comprises an antiwrinkle product.

19. A kit according to claim 17, wherein the product comprises a peeling product.

20. A kit according to claim 17, wherein the product comprises a product configured to stimulate renewal of epidermis.

21. A kit according to claim 17, wherein the product comprises a slimming product.

22. A kit according to claim 17, wherein the product comprises an anti-acne product.

23. A kit according to claim 17, wherein the product comprises a depilatory product.

24. A kit according to claim 17, wherein the product comprises a sunscreen.

25. A kit according to claim 17, wherein the product comprises at least one of a makeup and a care product.

26. A kit according to claim 11, further comprising a tube including a break-off end and an application end, the substance being contained in the tube.

27. A kit enabling an individual's level of tolerance relative to application of a product on skin of the individual to be determined, comprising:
a substance that contains at least one agent for stimulating the peripheral nervous system; and
a member configured to exert a mechanical action on the skin that lowers a barrier function thereof relative to the substance, and
an applicator configured to apply the substance to the skin and distinct from the member configured to exert the mechanical action on the skin.

28. A kit according to claim 27, wherein the member configured to exert the mechanical action on the skin is adhesive.

29. A kit according to claim 27, wherein the member configured to exert the mechanical action on the skin is abrasive.

30. A kit according to claim 27, wherein the substance comprises the product to be applied to the skin after evaluating the individual's level of tolerance to said product.

31. A kit according to claim 27, wherein the substance is different from the product to be applied to the skin after evaluating the individual's level of tolerance to said product.

32. A kit according to claim 27, wherein the substance contains at least one of capsaicin, lactic acid, alcohol and sodium chloride.

33. A kit according to claim 27, wherein the substance is contained in single-application packaging.

34. A kit according to claim 27, including a lotion for rinsing the test zone.

35. A kit according to claim 27, further including the substance that is to be applied to the skin.

36. A kit according to claim 27, wherein the product comprises an antiwrinkle product.

37. A kit according to claim 27, wherein the product comprises a peeling product.

38. A kit according to claim 27, wherein the product comprises a product configured to stimulate renewal of epidermis.

39. A kit according to claim 27, wherein the product comprises a slimming product.

40. A kit according to claim 27, wherein the product comprises an anti-acne product.

41. A kit according to claim 27, wherein the product comprises a depilatory product.

42. A kit according to claim 27, wherein the product comprises a sunscreen.

43. A kit according to claim 27, wherein the product comprises at least one of a makeup and a care product.

44. A kit according to claim 27, further comprising a tube including a break-off end and an application end, the substance being contained in the tube.

45. A method of prescribing a cosmetic, comprising:
evaluating a degree of tolerance of the skin by implementing the method according to claim 1; and
prescribing at least one of a cosmetic and a dosage as a function of a result of the evaluation.
